(19) United States
(12) Patent Application Publication
(10) Pub. No.: US 2003/0147830 A1
(43) Pub. Date: Aug. 7, 2003

(54) Topical skin and/or hair compositions containing protein

(75) Inventors: Jennifer Elizabeth Phillips, Cincinnati, OH (US); Bradley Steven Resch, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/061,039

(22) Filed: Jan. 30, 2002

(51) Int. Cl. A61K 7/06; A61K 7/11

(52) U.S. Cl. 424/70.14; 424/70.13

(57) ABSTRACT

Topical personal care, especially skin care, compositions containing at least one protein and at least one organic powder in an emulsion carrier. Such compositions are useful for providing a skin-tightening effect to the skin while retaining good aesthetics.

### FIELD OF INVENTION

The present invention relates to the field of topical personal care compositions containing a protein. The present invention also relates to the field of topical personal care compositions containing organic powder materials.

### BACKGROUND OF THE INVENTION

Skin is subject to insults by many extrinsic and intrinsic factors. Extrinsic factors include ultraviolet radiation (e.g., from sun exposure), environmental pollution, wind, heat, low humidity, harsh surfactants, abrasives, and the like. Intrinsic factors include chronological aging and other biochemical changes from within the skin. Whether extrinsic or intrinsic, these factors result in visible signs of skin aging and environmental damage, such as wrinkling and other forms of roughness (including increased pore size, flaking and skin lines), and other histological changes associated with skin aging or damage. To many people, skin wrinkles are a reminder of the disappearance of youth. As a result, the elimination of wrinkles has become a booming business in youth-conscious societies. Treatments range from cosmetic creams and moisturizers to various forms of cosmetic surgery.

Numerous skin care actives have been described in the art as being useful for regulating skin condition, including regulating fine lines, wrinkles and other forms of uneven or rough surface texture associated with aged or photodamaged skin.

Many of these actives are known to reduce the appearance of wrinkles after chronic use after a lengthy period of time. However, consumers are typically reluctant to use a product that does not provide a short-term, recognizable benefit. Therefore, the need exists for skin care formulations that provide a “signal” to the consumer that the product is working. Skin sensations caused by the application of skin care products, such as a localized and instantaneous tightening feel to the skin, warmth to the skin, and/or skin tingling are consumer preferable signals/indications that the skin care product is “working.”

Furthermore, consumers desire skin care products that generally exhibit a smooth, non-tacky, pleasant after feel and leave the skin feeling moisturized.

Consequently, recent trends in the personal care industry have created a surge of interest around the use of proteins (in skin care products) for their excellent moisturization, film-formation, and skin tightening properties. Proteins are long-chain, high molecular weight polymers consisting of amino acids that are joined by peptide bonds. These proteins can be derived from animal sources or from vegetable sources and are commercially available with a wide range of physical, chemical, and structural properties.

However, the inclusion of proteins, especially at higher levels, in skin care compositions often results in compositions with undesirable aesthetics. For example, protein-containing compositions are often sticky and/or tacky after application to the skin. Additionally, the desired tightening signal may be perceived by the consumer as drying and be perceived to correspond to a loss in skin moisture. Importantly, as the level of protein in the formulation increases, the undesirable aesthetics become more significant.

One method of reducing the undesirable aesthetics is to add oils to the protein-containing formulation. The addition of oils does reduce the sticky/tacky skin feel. However, the addition of oils often negates the tightening signal and results in formulations with an undesirable greasy and/or heavy after-feel.

Similarly, the introduction of humectants into the protein-containing formulations was successful in reducing the perception of drying/loss of skin moisture. However, the undesirable sticky/tacky skin feel was further increased by the humectants.

Based on the foregoing, there is a continuing need to formulate skin care compositions containing proteins and having an improved skin tightening signal while maintaining good skin feel and aesthetics.

Organic cosmetic powders are commonly used in skin care and hair care products to act as lubricants and give the formula a more silky, smooth feel. When used in topical personal care compositions, such powders function like microscopic ball bearings on the skin and/or hair, thereby creating a lubricated, soft feel on the skin. The basic mode of action of the powder occurs because the size of the particle is greater than the thickness of the product film on skin. Thus, when the product is rubbed against the skin, the powders are felt against the skin instead of the remainder of the product composition. Powders known in the art may be spherical, sphere-like, or irregularly shaped.

### SUMMARY OF THE INVENTION

The present invention relates to topical personal care compositions containing at least one protein that is a hydrolyzed or partially-hydrolyzed protein and at least one organic powder in a topical carrier.

The compositions of the present invention contain:

a) at least one protein selected from hydrolyzed proteins, partially-hydrolyzed proteins, and mixtures thereof;

b) at least one organic powder; and

c) a dermatologically acceptable carrier; wherein the carrier is in the form of an emulsion.

All documents cited are, in relevant part, incorporated herein by reference; the citation of any document is not to be construed as an admission that it is prior art with respect to the present invention.

### DETAILED DESCRIPTION OF THE INVENTION

It has surprisingly been found that the addition of organic powders to formulations containing at least one hydrolyzed or even partially-hydrolyzed protein makes it possible to obtain topical personal care formulations with a skin tightening signal while maintaining good skin feel and good aesthetics.

While the specification concludes with the claims particularly pointing and distinctly claiming the invention, it is believed that the present invention will be better understood from the following description.

All percentages and ratios used herein are by weight of the total composition and all measurements made are at 25° C., unless otherwise designated.

The term “ambient conditions” as used herein refers to surrounding conditions under about one atmosphere of pressure, at about 50% relative humidity, and at about 25° C. unless otherwise specified.

The compositions of the present invention can include, consist essentially of, or consist of, the components of the present invention as well as other ingredients described herein. As used herein, “consisting essentially of” means that the composition or component may include additional ingredients, but only if the additional ingredients do not materially alter the basic and novel characteristics of the claimed compositions or methods.

All percentages, parts and ratios are based upon the total weight of the personal care compositions of the present invention, unless otherwise specified. All such weights as they pertain to listed ingredients are based on the active level and, therefore, do not include carriers or by-products that may be included in commercially available materials, unless otherwise specified.

The term “keratinous tissue,” as used herein, refers to keratin-containing layers disposed as the outermost protective covering of mammals (e.g., humans, dogs, cats, etc.) which includes, but is not limited to, skin, lips, hair, toenails, fingernails, cuticles, hooves, etc.

The term “dermatologically-acceptable,” as used herein, means that the compositions or components thereof so described are suitable for use in contact with mammalian keratinous tissue without undue toxicity, incompatibility, instability, allergic response, and the like.

The term “safe and effective amount” as used herein means an amount of a compound or composition sufficient to significantly induce a positive benefit, preferably a positive keratinous tissue appearance or feel benefit, or positive hair appearance or feel benefit, including independently or in combinations the benefits disclosed herein, but low enough to avoid serious side effects, i.e., to provide a reasonable benefit to risk ratio, within the scope of sound judgment of the skilled artisan.

The term “sagging” as used herein means the laxity, slackness, or the like condition of skin that occurs as a result of loss of, damage to, alterations to, and/or abnormalities in dermal elastin.

The terms “smoothing” and “softening” as used herein mean altering the surface of the keratinous tissue such that its tactile feel is improved.

“Signs of skin aging” include, but are not limited to, all outward visibly and tactilely perceptible manifestations as well as any other macro or micro effects due to skin aging. Such signs may be induced or caused by intrinsic factors or extrinsic factors, e.g., chronological aging and/or environmental damage. These signs may result from processes which include, but are not limited to, the development of textural discontinuities such as wrinkles and coarse deep wrinkles, skin lines, crevices, bumps, large pores (e.g., associated with adnexal structures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including undereye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues, especially those proximate to the skin.

The compositions of the present invention are also useful for regulating the condition of skin and especially for regulating keratinous tissue condition. Regulation of skin condition, namely mammalian and in particular human skin condition, is often required due to conditions which may be induced or caused by factors internal and/or external to the body. Examples include, environmental damage, radiation exposure (including ultraviolet radiation), chronological aging, menopausal status (e.g., post-menopausal changes in skin), stress, diseases, etc.

As used herein, prophylactically regulating skin condition includes delaying, minimizing and/or preventing visible and/or tactile discontinuities in skin (e.g., texture irregularities in the skin which may be detected visually or by feel).

As used herein, therapeutically regulating skin condition includes ameliorating, e.g., diminishing, minimizing and/or effacing, discontinuities in skin.

The compositions of the present invention may also provide additional benefits, including absence of significant (consumer-unacceptable) skin irritation and good aesthetics.

The compositions of the present invention contain at least one protein selected from hydrolyzed proteins, partially-hydrolyzed proteins and mixtures thereof; at least one cosmetic organic powder, and a topical emulsion carrier. The compositions herein may also include a wide variety of other ingredients. The compositions of the present invention are described in detail hereinafter.

Hydrolyzed and Partially Hydrolyzed Proteins

The compositions of the present invention may contain a safe and effective amount of at least one protein selected from the group of hydrolyzed proteins, partially-hydrolyzed proteins, and mixtures thereof. Preferably, the protein or proteins are present in concentrations ranging from about 0.0001% to about 40% by weight, more preferably from about 0.001% to about 10% by weight, and most preferably from about 0.001% to about 5%, by weight of the composition.

Proteins are long-chain, high molecular weight polymers consisting of amino acids that are joined by peptide bonds. The term “protein” as used in this invention refers to a peptide chain having at least two amino acid residues, preferably at least five, and more preferably having more than one hundred amino acid residues.

The proteins useful for film-formation in the present invention are hydrolyzed and/or partially hydrolyzed proteins. The term “hydrolyzed protein” refers to the product of the hydrolysis of homogeneous or heterogeneous proteins, or their respective components, derivatives or combinations thereof. Hydrolysis typically involves the breaking of peptide bonds that join the amino acids together. By breaking these peptide bonds, the size of the natural (typically water-insoluble) polymer is reduced from a molecular weight in the millions to a molecular weight in the thousands. Methods for producing hydrolyzed proteins from the vegetable, animal, or synthetic based protein sources include, but are not limited to: 1) acid hydrolysis; 2) alkali hydrolysis; and 3) enzyme hydrolysis using a suitable protease. In alkaline hydrolysis, the peptide bonds are non-specifically opened in accordance with the rules of statistics. Since the carboxy groups of the peptides are present as salts during the hydrolysis while the amino groups are unprotected and can be partly eliminated, a hydrolyzate is obtained in which the polypeptides contain a larger number of carboxy groups than amino groups. Acidic hydrolysis also results in non-specific opening of the peptide bonds. In contrast to alkaline hydrolysis, however, the amino groups are present in salt form during the acidic degradation while the carboxy groups are present in free form but have a considerably higher stability than the unprotected amino groups. Protein hydrolyzates that have been prepared by enzymatic methods involve enzymes acting specifically on the peptide bond. The average molecular weight can be adjusted throughout the reaction conditions for all three hydrolysis processes. These methods, along with several others for preparing hydrolyzed proteins are well known in the art.

As used herein, “partially-hydrolyzed” refers to those proteins that are not completely hydrolyzed, yet have some degree (no matter how minor) of hydrolyzation.

These proteins may be chemically modified with quaternary groups, fatty groups, fatty alkyl quaternary groups, silicone groups, or may be a protein copolymer. This class of proteins does not include “native proteins” that exist in their original, perhaps biologically active, state.

The compositions of the present invention are not limited to the source of the hydrolyzed and/or partially-hydrolzyed protein. Non-limiting examples of sources of hydrolyzed and/or partially-hydrolyzed plant derived proteins which may be used in the invention, include: soya proteins, wheat proteins, almond protein, potato protein, oat proteins, pea proteins, sun flower proteins, corn proteins, cottonseed proteins, peanut proteins, and wheat germ protein. Other non-limiting examples include compounds containing hydrolyzed vegetable protein (and) hydrolyzed vegetable starch such as CROPEPTIDE W made by the company Croda; hydrolyzed vegetable protein polysiloxane copolymers such as CRODASONE W made by the company Croda; and hydrolyzed vegetable protein polyvinylpyrrolidone copolymers such as Hydrotriticum PVP made by the company Croda.

Non-limiting examples of sources of hydrolyzed and/or partially-hydrolyzed animal derived proteins which may be used in the invention, include: milk proteins, such as β-lactoglobulin, casein, or whey; serum proteins, such as horse serum; placental proteins; albumen; amylase; collagen; crystalline; cytochrome C; elastin; fibronectin; gelatin; gliadin; keratin; lipase; and serum albumin.

Preferably the protein has an average molecular weight of at least 75,000 Daltons, more preferably greater than 150,000 Daltons.

Most preferably the protein is a high molecular weight (average molecular weight of great than 150,000 Daltons) polypeptide.

The protein is preferably water soluble, and may be a natural, plant (vegetable) protein, or animal derived protein, as well as synthetic protein.

Mixtures of more than one protein may also be used. Hydrolyzed and partially-hydrolyzed proteins suitable for the compositions of the present invention are commercially available.

Organic Cosmetic Powder

The compositions of the present invention may contain a safe and effective amount of at least one organic cosmetic powder. Preferably, the powder or powders are present in concentrations ranging from about 0.0001% to about 5%, more preferably from about 0.1% to about 2.5%, and most preferably from about 0.25% to about 2%, by weight of the composition.

Cosmetic powders useful in the present invention include spherical, sphere-like, platelet, and irregularly shaped powders with average particle sizes ranging from about 0.01 microns to about 100 microns. Preferred cosmetic powders include spherical, sphere-like, and platelet shaped powders with average particle sizes ranging from about 0.1 to about 50 microns. More preferred average particle sizes range from about 0.1 to about 20 microns. Spherical or sphere-like powders are preferred.

Primary particle size can be determined using the ASTM Designation E20-85 “Standard Practice for Particle Size Analysis of Particulate Substances in the Range of 0.2 to 75 Micrometers by Optical Microscopy”, ASTM Volume 14.02, 1993, incorporated herein by reference.

Non-limiting examples of cosmetic powders useful in the present invention include powders made from boron nitride, cellulose triacetate, ethylene acrylic acid copolymer, mica, sericite, nylon-6, nylon-12, polymethylmethacrylate, polyethylene, polymethylsilsesquioxane, polytetraflouroethylene (“PTFE”), aluminum starch octenylsuccinate, polypropylene, L-lauroyl lysine, silicone resin, silk, and talc.

The cosmetic powders may also be coated with a surface coating to modify the behavior and sensory characteristics of the powder. Non-limiting examples of suitable coating materials include silicones, lecithin, amino acids, metal soaps, polyethylene, and collagen.

Preferred spherical and sphere-like cosmetic powders useful in the present invention include powders made from polytetraflouroethylene, polyethylene, polypropylene, nylon-12, polymethylsilsesquioxane silicone polymer, and mixtures thereof. More preferred are powders made from polymethylsilsesquioxane, nylon-12, polytetraflouroethylene, and mixtures thereof.

Dermatologically Acceptable Carrier

The compositions of the present invention may contain a safe and effective amount of a dermatologically acceptable carrier, wherein the carrier is in the form of an emulsion. The carrier ensures that the protein and organic powder of the present invention can be applied to and distributed evenly over the selected target at an appropriate concentration.

The carrier may contain one or more dermatologically acceptable solid, semi-solid or liquid fillers, diluents, solvents, extenders and the like. The carrier may be solid, semi-solid or liquid. Preferred carriers are substantially liquid. The carrier itself can be inert or it can possess dermatological benefits of its own. Concentrations of the carrier can vary with the carrier selected and the intended concentrations of the other components.

The characteristics of the emulsion carrier utilized in the present invention depend on the type of product form desired for the composition. The topical compositions useful in the subject invention may be made into a wide variety of product forms such as are known in the art. These include, but are not limited to, lotions, creams, sticks, sprays, ointments, pastes, mousses and cosmetics (e.g., solid, semi-solid, or liquid make-up, including foundations, eye-makeup, pigmented or non-pigmented lip treatments, e.g., lipsticks, and the like).

The emulsion carrier of the present invention contains a hydrophilic phase comprising a hydrophilic component, e.g., water or other hydrophilic diluent, and a hydrophobic phase comprising a hydrophobic component, e.g., a lipid, oil or oily material. As well known to one skilled in the art, the hydrophilic phase will be dispersed in the hydrophobic phase, or vice versa, to form respectively hydrophilic or hydrophobic dispersed and continuous phases, depending on the composition ingredients. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The emulsion may be or comprise (e.g., in a triple or other multi-phase emulsion) an oil-in-water emulsion or a water-in-oil emulsion such as a water-in-silicone emulsion. Oil-in-water emulsions typically comprise from about 1% to about 50% (preferably about 1% to about 30%) of the dispersed hydrophobic phase and from about 1% to about 98% (preferably from about 40% to about 90%) of the continuous hydrophilic phase; water-in-oil emulsions typically comprise from about 1% to about 98% (preferably from about 40% to about 90%) of the dispersed hydrophilic phase and from about 1% to about 50% (preferably about 1% to about 30%) of the continuous hydrophobic phase. The emulsion may also comprise a gel network, such as described in G. M. Eccleston, Application of Emulsion Stability Theories to Mobile and Semisolid O/W Emulsions, Cosmetics & Toiletries, Vol. 101, November 1996, pp. 73-92, incorporated herein by reference. Preferred emulsions are further described below.

Nonlimiting examples of hydrophilic diluents useful herein, include water, organic hydrophilic diluents such as lower monovalent alcohols (e.g., C₁-C₄) and low molecular weight glycols and polyols, including propylene glycol, polyethylene glycol (e.g., Molecular Weight 200-600 g/mole), polypropylene glycol (e.g., Molecular Weight 425-2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof. Water is a preferred diluent.

The topical compositions of the present invention, including but not limited to lotions and creams, may comprise a dermatologically acceptable emollient. Such compositions preferably contain from about 2% to about 50% of the emollient. Emollients tend to lubricate the skin, increase the smoothness and suppleness of the skin, prevent or relieve dryness of the skin, and/or protect the skin. Emollients are typically water-immiscible, oily or waxy materials. A wide variety of suitable emollients are known and may be used herein. Sagarin, Cosmetics, Science and Technology, 2nd Edition, Vol. 1, pp. 32-43 (1972), incorporated herein by reference, contains numerous examples of materials suitable as an emollient.

Lotions according to the present invention typically comprise from about 1% to about 20%, preferably from about 5% to about 10%, of emollient; from about 50% to about 90%, preferably from about 60% to about 80%, water. Creams according to the present invention typically comprise from about 5% to about 50%, preferably from about 10% to about 20%, of emollient; and from about 45% to about 85%, preferably from about 50% to about 75%, water.

Compositions of this invention useful for cleansing (“cleansers”) are formulated with a suitable carrier, e.g., as described above, and preferably contain one or more dermatologically acceptable surfactants in an amount which is safe and effective for cleansing. Preferred compositions contain from about 1% to about 90%, more preferably from about 5% to about 10%, of a dermatologically acceptable surfactant. The surfactant is suitably selected from anionic, cationic, nonionic, zwitterionic, amphoteric and ampholytic surfactants, as well as mixtures of these surfactants. Examples of a broad variety of surfactants useful herein are described in McCutcheon's Detergents and Emulsifiers, North American Edition (1986), published by Allured Publishing Corporation, which is incorporated herein by reference in its entirety. The cleansing compositions can optionally contain, at their art-established levels, other materials which are conventionally used in cleansing compositions.

The physical form of the cleansing compositions is not critical. The compositions can be, for example, formulated as toilet bars, liquids, shampoos, bath gels, hair conditioners, hair tonics, pastes, or mousses.

As used herein, the term “foundation” refers to a liquid, semi-liquid, semi-solid, or solid skin cosmetic which includes, but is not limited to lotions, creams, gels, pastes, cakes, and the like. Typically the foundation is used over a large area of the skin, such as over the face, to provide a particular look. Foundations are typically used to provide an adherent base for color cosmetics such as rouge, blusher, powder and the like, and tend to hide skin imperfections and impart a smooth, even appearance to the skin.

The compositions of the present invention are preferably formulated to have a pH of 10.5 or below. The pH values of these compositions preferably range from about 2 to about 10.5, more preferably from about 3 to about 8, even more preferably from about 5 to about 8.

Non-limiting examples of emulsion carriers useful herein, include oil-in-water, water-in-oil, water-in-silicone, water-in-oil-in-water, and oil-in-water-in-silicone emulsions. A preferred dermatologically acceptable carrier is in the form of an oil-in-water emulsion. As will be understood by the skilled artisan, a given component will distribute primarily into either the water or oil/silicone phase, depending on the water solubility/dispersibility of the component in the composition.

The dermatologically acceptable carrier may contain other ingredients, such as thickening agents, structuring agents, silicone elastomers, and mixtures thereof (more fully discussed below) in order to modify the viscosity and/or feel of the composition.

### Optional Ingredients

The compositions of the present invention may contain one or more additional skin care components. In a preferred embodiment, where the composition is to be in contact with human keratinous tissue, the additional components should be suitable for application to keratinous tissue, that is, when incorporated into the composition they are suitable for use in contact with human keratinous tissue without undue toxicity, incompatibility, instability, allergic response, and the like within the scope of sound medical judgment.

The CTFA Cosmetic Ingredient Handbook, Second Edition (1992) describes a wide variety of nonlimiting cosmetic and pharmaceutical ingredients commonly used in the personal care industry, which are suitable for use in the compositions of the present invention.

In any embodiment of the present invention, however, the actives useful herein can be categorized by the benefit they provide or by their postulated mode of action. However, it is to be understood that the actives useful herein can in some instances provide more than one benefit or operate via more than one mode of action. Therefore, classifications herein are made for the sake of convenience and are not intended to limit the active to that particular application or applications listed.

Non-Hydrolyzed Proteins

In addition to the hydrolyzed and/or partially-hydrolyzed proteins of the present invention, non-hydrolyzed proteins or native proteins may also be included. Non-limiting examples of plant derived non-hydrolyzed proteins useful herein, include: soya proteins, wheat proteins, almond protein, potato protein, oat proteins, pea proteins, sun flower proteins, corn proteins, cottonseed proteins, peanut proteins, and wheat germ protein. Non-limiting examples of animal derived non-hydrolyzed proteins useful herein, include: milk proteins, such as lactoglobulin, casein, or whey; serum proteins, such as horse serum; placental proteins; albumen; amylase; collagen; crystalline; cytochrome C; elastin; fibronectin; gelatin; gliadin; keratin; lipase; and serum albumin.

Silicone Elastomers

The compositions of the present invention may contain a silicone elastomer. When present, the composition preferably comprises from about 0.1% to about 30%, more preferably from about 1% to about 20%, and even more preferably, from about 2% to about 10%, by weight of the composition, of a silicone elastomer component.

The compositions of the present invention may include an emulsifying crosslinked organopolysiloxane elastomer, a non-emulsifying crosslinked organopolysiloxane elastomer, or a mixture thereof. The term “non-emulsifying,” as used herein, defines crosslinked organopolysiloxane elastomers from which polyoxyalkylene units are absent. The term “emulsifying,” as used herein, means crosslinked organopolysiloxane elastomers having at least one polyoxyalkylene (e.g., polyoxyethylene or polyoxypropylene) unit.

No specific restriction exists as to the type of curable organopolysiloxane composition which can serve as starting material for the crosslinked organopolysiloxane elastomer.

Non-limiting examples of emulsifying elastomers include polyoxyalkylene modified elastomers formed from divinyl compounds, particularly siloxane polymers with at least two free vinyl groups, reacting with Si-H linkages on a polysiloxane backbone. Preferably, the elastomers are dimethyl polysiloxanes crosslinked by Si-H sites on a molecularly spherical MQ resin. Emulsifying crosslinked organopolysiloxane elastomer can notably be chosen from the crosslinked polymers described in U.S. Pat. Nos. 5,412,004 (issued May 2, 1995); 5,837,793 (issued Nov. 17, 1998); and 5,811,487 (issued Nov. 22, 1998). In addition, an emulsifying elastomer comprised of dimethicone copolyol crosspolymer (and) dimethicone is available from Shin Etsu under the tradename KSG-21.

Non-limiting examples of non-emulsifying elastomers are dimethicone/vinyl dimethicone crosspolymers. Such dimethicone/vinyl dimethicone crosspolymers are supplied by a variety of suppliers including Dow Corning (DC 9040 and DC 9041), General Electric (SFE 839), Shin Etsu (KSG-15, 16, 18 [dimethicone/phenyl vinyl dimethicone crosspolymer]), and Grant Industries (GRANSIL™ line of elastomers). Cross-linked organopolysiloxane elastomers useful in the present invention and processes for making them are further described in U.S. Pat. No. 4,970,252 to Sakuta, et al., issued Nov. 13, 1990; U.S. Pat. No. 5,760,116 to Kilgour, et al., issued Jun. 2, 1998; U.S. Pat. No. 5,654,362 to Schulz, Jr., et al. issued Aug. 5, 1997. Additional crosslinked organopolysiloxane elastomers useful in the present invention are disclosed in Japanese Patent Application JP 61-18708, assigned to Pola Kasei Kogyo KK.

Commercially available elastomers preferred for use herein are Dow Corning's 9040 silicone elastomer blend, Shin Etsu's KSG-21, and mixtures thereof

Structuring Agent

The compositions of the present invention, in some embodiments, may further include a structuring agent. Compositions of this invention may contain from about 0.1% to about 20%, more preferably from about 0.1% to about 10%, still more preferably from about 0.5% to about 9%, of one or more structuring agents.

Preferred structuring agents for use herein are those having an HLB of from about 1 to about 8 and having a melting point of at least about 45° C. Non-limiting examples of structuring agents useful in compositions of the present invention include stearic acid, palmitic acid, stearyl alcohol, cetyl alcohol, behenyl alcohol, stearic acid, palmitic acid, the polyethylene glycol ether of stearyl alcohol having an average of about 1 to about 5 ethylene oxide units, the polyethylene glycol ether of cetyl alcohol having an average of about 1 to about 5 ethylene oxide units, and mixtures thereof.

Thickening Agents

The compositions of the present invention, in some embodiments, may further include one or more thickening agents. When present, the composition preferably includes from about 0.1% to about 5%, more preferably from about 0.1% to about 4%, and still more preferably from about 0.25% to about 3%, by weight of the composition of the thickening agent.

Nonlimiting examples of thickening agents useful herein include carboxylic acid polymers such as the carbomers (such as those commercially available under the tradename Carbopol® 900 series from B. F. Goodrich; e.g., Carbopol® 954). Other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as Carbopol® 1342, Carbopol® 1382, PEMULEN TR-1, and PEMULEN TR-2, from B. F. Goodrich.

Other nonlimiting examples of thickening agents include crosslinked polyacrylate polymers including both cationic and nonionic polymers.

Still other nonlimiting examples of thickening agents include the polyacrylamide polymers, especially nonionic polyacrylamide polymers including substituted branched or unbranched polymers. More preferred among these polyacrylamide polymers is the nonionic polymer given the CTFA designation polyacrylamide and isoparaffin and laureth-7, available under the Tradename Sepigel 305 from Seppic Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500W, SSSA100H, from Lipo Chemicals, Inc., (Patterson, N.J.).

Another nonlimiting class of thickening agents useful herein are the polysaccharides. Nonlimiting examples of polysaccharide gelling agents include those selected from cellulose, and cellulose derivatives. Preferred among the alkyl hydroxyalkyl cellulose ethers is the material given the CTFA designation cetyl hydroxyethylcellulose, which is the ether of cetyl alcohol and hydroxyethylcellulose, sold under the tradename Natrosol® CS Plus from Aqualon Corporation (Wilmington, Del.). Other useful polysaccharides include scleroglucans which are a linear chain of (1-3) linked glucose units with a (1-6) linked glucose every three units, a commercially available example of which is Clearogel™ CS11 from Michel Mercier Products Inc. (Mountainside, N.J.).

Another nonlimiting class of thickening agents useful herein are the gums. Nonlimiting examples of gums useful herein include hectorite, hydrated silica, xantham gum, and mixtures thereof.

Vitamins

Non-limiting examples of vitamins useful herein include vitamin B3 compounds (such as niacinamide, tocopherol nicotinate), vitamin C (such as magnesium ascorbyl phosphate, ascorbyl glucoside), Vitamin A or derivatives (such as retinol, retinyl palmitate, retinyl acetate, retinyl propionate), Vitamin B5 or derivatives (such as panthenol, pantothenoic acid), Vitamin E or derivatives (such as tocopherol, tocopherol acetate), or Vitamin D3 or derivatives.

Vitamin B3 Compounds

The compositions of the present invention may include, in some embodiments, a vitamin B₃ compound. Salts of the vitamin B₃ compound are also useful herein. When present, the composition preferably includes from about 0.01% to about 50%, more preferably from about 0.1% to about 10%, by weight of the composition, of the vitamin B₃ compound.

Non-limiting examples of vitamin B3 compounds useful herein include niacinamide, tocopherol nicotinate, and mixtures thereof.

Zeolites

Non-limiting examples of zeolites useful herein include natural zeolites such as analcite, chabazite, heulandite, natrolite, stilbite, and thomosonite; and synthetic zeolites such as those made by the gel process (sodium silicate and alumina) or a clay process (kaolin), which forms a matrix to which the zeolite is added.

Peptides

Peptides, including but not limited to, di-, tri-, tetra-, and pentapeptides and derivatives thereof, may be included in the compositions of the present invention in amounts that are safe and effective. Non-limiting examples of peptides and peptide derivatives useful herein include; Carnosineg (beta-ala-his), gly-his-lys, arg-lys-arg, his-gly-gly, palmitoyl-gly-his-lys (which may be purchased as Biopeptide CL®, 100 ppm commercially available from Sederma, France), Peptide CK (arg-lys-arg), PEPTIDE CK+ (ac-arg-lys-arg-NH₂), and a copper derivative of his-gly-gly sold commercially as IAMIN, from Sigma (St. Louis, Mo.). Tetrapeptides and pentapeptides (such as palmitoyl-lys-thr-thr-lys-ser commercially available from Sederma France) are also suitable for use herein.

When included in the present compositions, peptides are preferably included in amounts of from about 1×10⁻⁶% to about 10%, more preferably from about 1×10⁻⁶% to about 0.1%, by weight of the composition.

Sunscreen Actives

The compositions of the subject invention may contain a sunscreen active. As used herein, “sunscreen active” includes both sunscreen agents and physical sunblocks.

Inorganic sunscreens useful herein include the following metallic oxides; titanium dioxide having an average primary particle size of from about 15 nm to about 100 nm, zinc oxide having an average primary particle size of from about 15 nm to about 150 nm, iron oxide having an average primary particle size of from about 15 nm to about 500 nm, and mixtures thereof. When used herein, the inorganic sunscreens are present in the amount of from about 0.1% to about 20%, preferably from about 0.5% to about 10%, by weight of the composition.

A wide variety of conventional organic sunscreen actives are suitable for use herein. Sagarin, Vol. 102 pages 21 et seq., of Cosmetics and Toiletries (1987), discloses numerous suitable actives. Nonlimiting examples of organic sunscreen actives useful herein include octylsalicylate, 2-Phenylbenzimidazole-5-sulphonic acid salts, Salts of Terephthalylidene Dicamphor sulfonic acid, octocrylene, octylmethoxycinnamate, avobenzone, and mixtures thereof.

When present in compositions of the present invention, a safe and effective amount of the organic sunscreen active is used, typically from about 1% to about 20%, more typically from about 2% to about 10% by weight of the composition.

Terpene Alcohols

The topical compositions of the present invention may, in some embodiments, contain a safe and effective amount of a terpene alcohol such as phytantriol, phytantriol derivatives, farnesol, farnesol derivatives, and mixtures thereof. When included in compositions of the present invention, the terpene alcohol is preferably is included in an amount from about 0.001% to about 50% by weight of the composition, more preferably from about 0.01% to about 20%, by weight of the composition.

Desquamation Actives

A safe and effective amount of a desquamation active may be added to the compositions of the present invention, preferably from about 0.1% to about 10%, more preferably from about 0.2% to about 5%, by weight of the composition. Non-limiting examples of desquamation systems useful herein include; a combination of sulfhydryl compounds and zwitterionic surfactants; and a combination of salicylic acid and zwitterionic surfactants.

Anti-Acne Actives

The compositions of the present invention may contain a safe and effective amount of one or more anti-acne actives. Examples of useful anti-acne actives include resorcinol, sulfur, salicylic acid, benzoyl peroxide, erythromycin, zinc, etc.

Anti-Wrinkle Actives/Anti-Atrophy Actives

The compositions of the present invention may further contain a safe and effective amount of one or more anti-wrinkle actives or anti-atrophy actives. Non-limiting examples of anti-wrinkle/anti-atrophy actives suitable for use in the compositions of the present invention include hydroxy acids (e.g., alpha-hydroxy acids such as lactic acid and glycolic acid or beta-hydroxy acids such as salicylic acid and salicylic acid derivatives such as the octanoyl derivative), phytic acid, lipoic acid; lysophosphatidic acid, and skin peel agents.

Anti-Oxidants/Radical Scavengers

A safe and effective amount of an anti-oxidant/radical scavenger may be added to the compositions of the subject invention, preferably from about 0.1% to about 10%, more preferably from about 1% to about 5%, of the composition.

Non-limiting examples of anti-oxidants/radical scavengers useful herein include; ascorbic acid (vitamin C) and derivatives thereof; tocopherol (vitamin E) and derivatives thereof (e.g. tocopherol sorbate, tocopherol acetate); butylated hydroxy benzoic acids and their salts, 6-hydroxy-2,5,7,8-tetramethylchroman-2-carboxylic acid; sorbic acid and its salts; lipoic acid, amines (e.g., N,N-diethylhydroxylamine, amino-guanidine); tea extracts; grape skin/seed extracts; and mixtures thereof.

Flavonoids

The compositions of the present invention may optionally contain a flavonoid compound. Flavonoids are broadly disclosed in U.S. Pat. Nos. 5,686,082 and 5,686,367. Non-limiting examples of flavonoids useful herein include unsubstituted flavone, 7,2′-dihydroxy flavone, 3′,4′-dihydroxy naphthoflavone, 4′-hydroxy flavone, 5,6-benzoflavone, and 7,8-benzoflavone, unsubstituted isoflavone, daidzein (7,4′-dihydroxy isoflavone), 5,7-dihydroxy-4′-methoxy isoflavone, soy isoflavones (a mixture extracted from soy), and mixtures thereof.

When present, the flavonoid compounds are preferably present in concentrations of from about 0.01% to about 20%, more preferably from about 0.1% to about 10%, by weight of the composition.

Anti-Inflammatory Agents

A safe and effective amount of an anti-inflammatory agent may be added to the compositions of the present invention, preferably from about 0.1% to about 10%, more preferably from about 0.5% to about 5%, of the composition.

Nonlimiting examples of “natural” anti-inflammatory agents that are useful herein include candelilla wax, bisabolol (e.g., alpha bisabolol), aloe vera, plant sterols (e.g., phytosterol), and mixtures thereof.

Additional anti-inflammatory agents useful herein include glycyrrhizinate compounds such as dipotassium glycyrrhizinate.

Anti-Cellulite Agents

The compositions of the present invention may also contain a safe and effective amount of an anti-cellulite agent. Non-limiting examples of anti-cellulite agents useful herein include xanthine compounds (e.g., caffeine, theophylline, theobromine, and aminophylline).

Topical Anesthetics

The compositions of the present invention may also contain a safe and effective amount of a topical anesthetic. Examples of topical anesthetic drugs include benzocaine, lidocaine, pharmaceutically acceptable salts thereof, and mixtures thereof.

Tanning Actives

The compositions of the present invention may contain a tanning active. When present, it is preferable that the compositions contain from about 0.1% to about 20%, more preferably from about 2% to about 7%, by weight of the composition, of the artificial tanning active.

A non-limiting example of a tanning active useful herein is dihydroxyacetone.

Skin Lightening Agents

The compositions of the present invention may contain a skin lightening agent. When used, the compositions preferably contain from about 0.1% to about 10%, more preferably from about 0.2% to about 5%, by weight of the composition, of a skin lightening agent. Non-limiting examples of skin lightening agents useful herein include those known in the art, including niacinamide, kojic acid, arbutin, ascorbic acid and derivatives thereof (e.g sodium ascorbyl phosphate), and extracts (e.g., mulberry extract, placental extract).

Skin Soothing and Skin Healing Actives

The compositions of the present invention may include a skin soothing or skin healing active. Skin soothing or skin healing actives suitable for use herein include panthenoic acid derivatives (including panthenol, dexpanthenol, ethyl panthenol), aloe vera, allantoin, bisabolol, and dipotassium glycyrrhizinate. A safe and effective amount of a skin soothing or skin healing active may be added to the present composition, preferably, from about 0.1% to about 30%, more preferably from about 0.5% to about 20%, by weight of the composition.

Conditioning Agent

Some embodiments of the present invention may further contain a conditioning agent selected from humectants, moisturizers, or skin conditioners. A variety of these materials can be employed and each can be present at a level of from about 0.01% to about 20%, more preferably from about 0.1% to about 10%, by weight of the composition. Nonlimiting examples of conditioning agents useful herein include hyaluronic acid, glycerin, panthenol, allantoin, and mixtures thereof. Also useful are various C₁-C₃₀ monoesters and polyesters of sugars and related materials.

Methods of Use

The compositions of the present invention are useful for regulating the condition of skin and/or hair while maintaining good stability. Regulating the condition of skin includes reducing the appearance of fine lines and/or wrinkles on the skin, reducing the appearance of eye bags and dark circles under the eys, sagging skin, scars/marks, dimples, pores, stretch marks, roughness, skin surface blemishes, frown lines, expression lines, rhytides, blemishes, photodamage, crevices, and/or unevenness.

### EXAMPLES

The following examples further describe and demonstrate embodiments within the scope of the present invention. The examples are given solely for the purpose of illustration and are not to be construed as limitations of the present invention, as many variations there of are possible without departing from the spirit and scope of the invention.

Making Instructions

The examples of the present invention may all be prepared by conventional means. The following general making instructions apply to all of the examples provided below:

In a suitable container, all water phase materials are combined. In a separate container, all oil phase materials are blended together. Then, both containers are heated to 75° C. Once both phases have reached appropriate temperature, the oil phase is added to the water phase and milled for approximately 5 minutes. The batch is then slowly cooled. If additional phases are to be added to the composition, these are added, with mixing, to the batch at a temperature of between 50° C. and 60° C. When the batch temperature reaches 35° C., the batch is milled for another 5 minutes and then transferred to appropriate containers.

### Examples 1a-1d

Topical Cream

| | % w/w | | | |
|---|---|---|---|---|
| Ingredient | 1a | 1b | 1c | 1d |
| Phase A | | | | |
| Water | q.s. 100% | q.s. 100% | q.s. 100% | q.s. 100% |
| Disodium EDTA | 0.1 | 0.1 | 0.1 | 0.1 |
| Dexpanthenol | 0.5 | 0.5 | 0.5 | 0.5 |
| Niacinamide | 2.0 | 2.0 | 2.0 | 2.0 |
| Phase B | | | | |
| Glycerine | 5.0 | 5.0 | 5.0 | 5.0 |
| Hydrolyzed wheat | 2.0 | | | |
| protein | | | | |
| Hydrolyzed soy protein | | 2.0 | | |
| Hydrolyzed collagen | | | 2.0 | |
| Hydrolyzed elastin | | | 1.0 | |
| Hydrolyzed potato | | | | 3.0% |
| protein | | | | |
| Phase C | | | | |
| Isohexadecane | 3.0 | 3.0 | 3.0 | 3.0 |
| Isopropyl Isostearate | 1.0 | 1.0 | 1.0 | 1.0 |
| Stearic Acid | 0.4 | 0.4 | 0.4 | 0.4 |
| Tocopherol Acetate | 0.5 | 0.5 | 0.5 | 0.5 |
| Cetearyl Glucoside | 0.2 | 0.2 | 0.2 | 0.2 |
| Cetyl Alcohol | 1.0 | 1.0 | 1.0 | 1.0 |
| Polymethyl- | 0.5 | 0.5 | 0.5 | 0.5 |
| silsesquioxane | | | | |
| Phase D | | | | |
| Sodium Hydroxide | Adjust pH | | | |
| Phase E | | | | |
| Sepigel 305 | 1.5 | 1.5 | 1.5 | 1.5 |

### Examples 2a-2d

Topical Cream

| | % w/w | | | |
|---|---|---|---|---|
| Ingredient | 2a | 2b | 2c | 2d |
| Phase A | | | | |
| Water | q.s. 100% | q.s. 100% | q.s. 100% | q.s. 100% |
| Disodium EDTA | 0.1 | 0.1 | 0.1 | 0.1 |
| Dexpanthenol | 0.5 | 0.5 | 0.5 | 0.5 |
| Niacinamide | 2.0 | 2.0 | 2.0 | 2.0 |
| Phase B | | | | |
| Glycerine | 10 | 8.0 | 5.0 | 5.0 |
| Hydrolyzed wheat | 1.0 | 1.0 | 1.0 | 1.0 |
| protein | | | | |
| Hydrolyzed potato | 2.0 | 2.0 | 2.0 | 2.0 |
| protein | | | | |
| Sodium Hyaluronate (1% | | 2.0 | | 1.0 |
| solution) | | | | |
| Butylene Glycol | | | 5.0 | 2.0 |
| Phase C | | | | |
| Isohexadecane | 3.0 | 3.0 | 3.0 | 3.0 |
| Isopropyl Isostearate | 1.0 | 1.0 | 1.0 | 1.0 |
| Stearic Acid | 0.4 | 0.4 | 0.4 | 0.4 |
| Tocopherol Acetate | 0.5 | 0.5 | 0.5 | 0.5 |
| Cetearyl Glucoside | 0.2 | 0.2 | 0.2 | 0.2 |
| Cetyl Alcohol | 1.0 | 1.0 | 1.0 | 1.0 |
| Polymethyl- | 0.5 | 0.5 | 0.5 | 0.5 |
| silsesquioxane | | | | |
| Phase D | | | | |
| Sodium Hydroxide | Adjust pH | | | |
| Phase E | | | | |
| Sepigel 305 | 1.5 | 1.5 | 1.5 | 1.5 |

### Examples 3a-3d

Topical Cream

| | % w/w | | | |
|---|---|---|---|---|
| Ingredient | 3a | 3b | 3c | 3d |
| Phase A | | | | |
| Water | q.s. 100% | q.s. 100% | q.s. 100% | q.s. 100% |
| Disodium EDTA | 0.1 | 0.1 | 0.1 | 0.1 |
| Dexpanthenol | 0.5 | 0.5 | 0.5 | 0.5 |
| Niacinamide | 2.0 | 2.0 | 2.0 | 2.0 |
| Phase B | | | | |
| Glycerine | 5.0 | 5.0 | 5.0 | 5.0 |
| Hydrolyzed wheat | 2.0 | 2.0 | 2.0 | 2.0 |
| protein | | | | |
| Hydrolyzed potato | 1.0 | 1.0 | 1.0 | 1.0 |
| protein | | | | |
| Phase C | | | | |
| Isohexadecane | 3.0 | 3.0 | 3.0 | 3.0 |
| Isopropyl Isostearate | 1.0 | 1.0 | 1.0 | 1.0 |
| Stearic Acid | 0.4 | 0.4 | 0.4 | 0.4 |
| Tocopherol Acetate | 0.5 | 0.5 | 0.5 | 0.5 |
| Cetearyl Glucoside | 0.2 | 0.2 | 0.2 | 0.2 |
| Cetyl Alcohol | 1.0 | 1.0 | 1.0 | 1.0 |
| Polymethyl- | 0.5 | 0.75 | | |
| silsesquioxane | | | | |
| Nylon-12 | | | 2.0 | |
| Polyethylene | | | | 1.0 |
| Phase D | | | | |
| Sodium Hydroxide | Adjust pH | | | |
| Phase E | | | | |
| Sepigel 305 | 1.5 | 1.5 | 1.5 | 1.5 |

### Examples 4a-4d

Topical Cream

| | % w/w | | | |
|---|---|---|---|---|
| Ingredient | 4a | 4b | 4c | 4d |
| Phase A | | | | |
| Water | q.s. 100% | q.s. 100% | q.s. 100% | q.s. 100% |
| Hydroxyethylcellulose | 0.5 | 0.5 | 0.5 | 0.5 |
| Propylene Glycol | 0.25 | 0.25 | 0.25 | 0.25 |
| Panthenol | 0.5 | 0.5 | 0.5 | 0.5 |
| Methyl Paraben | 0.2 | 0.2 | 0.2 | 0.2 |
| Hydrolyzed Wheat | 2.0 | 2.0 | 2.0 | 2.0 |
| Protein | | | | |
| Phase B | | | | |
| Glycerol Stearate | 4.0 | 4.0 | 4.0 | 4.0 |
| Stearic Acid | 2.0 | 2.0 | 2.0 | 2.0 |
| PPG-12 Myristyl Ether | 2.0 | 2.0 | 2.0 | 2.0 |
| Propionate | | | | |
| C12-15 Alkyl Benzoate | 2.0 | 2.0 | 2.0 | 2.0 |
| Dimethicone | 1.0 | 1.0 | 1.0 | 1.0 |
| Avocado Oil | 0.5 | 0.5 | 0.5 | 0.5 |
| Unsaponifiables | | | | |
| Tridecyl Stearate | 0.5 | 0.5 | 0.5 | 0.5 |
| Neopentyl Glycol | 0.5 | 0.5 | 0.5 | 0.5 |
| Dicaprylate/Dicaprate | | | | |
| Tridecyl Trimellitate | 0.2 | 0.2 | 0.2 | 0.2 |
| Squalane | 0.4 | 0.4 | 0.4 | 0.4 |
| Propyl Paraben | 0.1 | 0.1 | 0.1 | 0.1 |
| Polymethyl- | 0.75 | — | — | — |
| silsesquioxane | | | | |
| PTFE | — | 0.75 | — | — |
| Nylon-12 | — | — | 0.75 | — |
| Polymethylmethacrylate | — | — | — | 0.75 |

### Examples 5a-5d

Topical Cream

| | % w/w | | | |
|---|---|---|---|---|
| Ingredient | 5a | 5b | 5c | 5d |
| Phase A | | | | |
| Water | q.s. 100% | q.s. 100% | q.s. 100% | q.s. 100% |
| Carbopol 1382 | 0.25 | 0.25 | 0.25 | 0.25 |
| Butyelene Glycol | 1.0 | 1.0 | 1.0 | 1.0 |
| Glycerin | 2.5 | 2.5 | 2.5 | 2.5 |
| Sodium Hyaluronate | 1.0 | 1.0 | 1.0 | 1.0 |
| Methyl Paraben | 0.1 | 0.1 | 0.1 | 0.1 |
| Titanium Dioxide | 0.75 | 0.75 | 0.75 | 0.75 |
| Hydrolyzed Soy Protein | 1.5 | — | 1.5 | — |
| Hydrolylzed Elastin | — | 1.5 | — | 1.5 |
| Phase B | | | | |
| Isocetyl Stearate | 2.0 | 2.0 | 2.0 | 2.0 |
| PEG-100 Stearate | 2.0 | 2.0 | 2.0 | 2.0 |
| Glyceryl Stearate | 2.0 | 2.0 | 2.0 | 2.0 |
| Petrolatum | 1.0 | 1.0 | 1.0 | 1.0 |
| Cetearyl Alcohol | 1.0 | 1.0 | 1.0 | 1.0 |
| Isocetyl Alcohol | 1.0 | 1.0 | 1.0 | 1.0 |
| Cetyl Ricinoleate | 0.5 | 0.5 | 0.5 | 0.5 |
| Polyglyceryl-3 Beeswax | 0.5 | 0.5 | 0.5 | 0.5 |
| Cholesterol | 0.5 | 0.5 | 0.5 | 0.5 |
| Cetearyl Glucoside | 0.2 | 0.2 | 0.2 | 0.2 |
| Propyl Paraben | 0.2 | 0.2 | 0.2 | 0.2 |
| Dimethicone Copolyall | 0.5 | 0.5 | 0.5 | 0.5 |
| Dimethicone | 0.75 | 0.75 | 0.75 | 0.75 |
| polytetraflouroethylene | 0.5 | 0.5 | — | — |
| polymethylsilsesquioxane | — | — | 0.5 | 0.5 |
| Phase C | | | | |
| Sepigel 501 | 1.0 | 1.0 | 1.0 | 1.0 |

### Examples 6a-6b

Topical Cream

| | % w/w | | | |
|---|---|---|---|---|
| Ingredient | 6a | 6b | 6c | 6d |
| Phase A | | | | |
| Water | q.s. 100% | q.s. 100% | q.s. 100% | q.s. 100% |
| Glycerin | 2.5 | 2.5 | 2.5 | 2.5 |
| Butyelene Glycol | 1.0 | 1.0 | 1.0 | 1.0 |
| Methyl Paraben | 0.1 | 0.1 | 0.1 | 0.1 |
| Hydrolyzed Wheat Flour | 0.25 | 0.25 | 0.25 | 0.25 |
| Hydrolyzed Potato | 2.5 | — | — | — |
| Protein | | | | |
| Hydrolyzed Wheat | — | 2.5 | — | — |
| Protein | | | | |
| Hydrolyzed Soy Protein | — | — | 2.5 | — |
| Hydrolyzed Collagen | — | — | — | 2.5 |
| Phase B | | | | |
| Hydrogenated Poly- | 2.5 | 2.5 | 2.5 | 2.5 |
| isobutene | | | | |
| Mineral Oil | 2.0 | 2.0 | 2.0 | 2.0 |
| Glyceryl Stearate | 1.0 | 1.0 | 1.0 | 1.0 |
| Cetyl Alcohol | 1.0 | 1.0 | 1.0 | 1.0 |
| PEG-40 Stearate | 1.5 | 1.5 | 1.5 | 1.5 |
| Sorbitan Tristearate | 1.0 | 1.0 | 1.0 | 1.0 |
| Dimethicone | 1.5 | 1.5 | 1.5 | 1.5 |
| Cyclopentasiloxane | 1.0 | 1.0 | 1.0 | 1.0 |
| Nylon-12 | 1.0 | 1.0 | 1.0 | 1.0 |
| Whey Protein | 0.1 | 0.25 | 0.5 | 1.0 |
| (lactis proteinum) | | | | |
| Phase C | | | | |
| Sepigel 305 | 1.0 | 1.0 | 1.0 | 1.0 |

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A topical personal care composition comprising:
a) at least protein selected from the group consisting of hydrolyzed proteins, partially-hydrolyzed proteins, and mixtures thereof;
b) at least one organic powder; and
c) a dermatologically acceptable carrier; wherein the carrier is in the form of an emulsion.

2. A topical composition according to claim 1 wherein the composition comprises from about 0.0001 to about 40%, by weight of the composition, of a hydrolzyed protein.

3. A topical composition according to claim 2, wherein the composition comprises from about 0.001 to about 5%, by weight of the composition, of the protein.

4. A topical composition according to claim 1 wherein the protein is selected from the group consisting of plant derived proteins and mixtures thereof.

5. A topical composition according to claim 4 wherein the protein is a hydrolyzed protein and is selected from the group consisting of soya proteins, wheat proteins, almond proteins, potato proteins, oat proteins, pea proteins, sunflower proteins, corn proteins, cottonseed proteins, peanut proteins, wheat germ proteins, and mixtures thereof.

6. A topical composition according to claim 5 wherein the hydrolyzed protein is wheat protein.

7. A topical composition according to claim 1 wherein the protein is an animal protein selected from the group consisting of β-lactoglobulin, casein, whey, horse serum, placental proteins, albumen, amylase, collagen, crystalline, cytochrome C, elastin, fibronectin, gelatin, gliadin, keratin, lipase, serum albumin, and mixtures thereof.

8. A topical composition according to claim 1 wherein the protein is a high molecular weight polypeptide.

9. A topical composition according to claim 1 wherein the protein is water-soluble.

10. A topical composition according to claim 1 wherein the composition comprises from about 0.0001% to about 5%, by weight of the composition, of the organic powder.

11. A topical composition according to claim 10 wherein the composition comprises from about 0.25% to about 2%, by weight of the composition, of the organic powder.

12. A topical composition according to claim 1 wherein the organic powder is a surface treated organic powder.

13. A topical composition according to claim 1 wherein the organic powder is selected from the group consisting of spherical powders, sphere-like powders, platelet powders, and mixtures thereof.

14. A topical composition according to claim 13 wherein the organic powder is a spherical powder.

15. A topical composition according to claim 1 wherein the organic powder is made from a material selected from the group consisting of boron nitride, cellulose triacetate, ethylene acrylic acid copolymer, mica, sericite, nylon-6, nylon-12, polymethylmethacrylate, aluminum starch octenylsuccinate, polyethylene, polypropylene, polymethylsilesquioxane, polytetraflouroethylene, silicone resin, silk, and talc.

16. A topical composition according to claim 15 wherein the organic powder is made from a material selected from the group consisting of nylon-12, polytetraflouroethylene, polymethylsilesquioxane, and mixtures thereof.

17. A topical composition according to claim 1 wherein the organic powder has an average particle size of from about 0.01 to about 50 microns.

18. A topical composition according to claim 17 wherein the organic powder has an average particle size of from about 0.1 to about 50 microns.

19. A topical composition according to claim 1 wherein the composition further comprises a skin care active selected from the group consisting of vitamins, non-hydrolyzed proteins, zeolites, peptides, skin-lightening agents, sunscreen actives, terpene alcohols, desquamation actives, anti-acne actives, anti-wrinkle actives, anti-atrophy actives, anti-oxidants, flavanoids, anti-inflammatory agents, anti-cellulite, topical anesthetics, tanning actives, skin soothing actives, skin healing actives, conditioning agents, and mixtures thereof

20. A topical composition according to claim 1 wherein the emulsion is an oil-in-water emulsion.

21. A method of producing a skin-tightening sensation, said method comprising the step of:
a) topically applying to the skin of a mammal in need of such treatment, a safe and effective amount of a composition according to claim 1.

22. An oil-in-water emulsion skin care composition comprising:
a) from about 0.0001 to about 5%, by weight of the composition, of organic powder;
b) from about 0.001% to about 40%, by weight of the composition, of hydrolyzed protein;
c) a humectant; and
d) a skin care active selected from the group consisting of vitamins, zeolites, peptides, skin-lightening agents, sunscreen actives, non-hydrolyzed proteins, terpene alcohols, desquamation actives, anti-acne actives, anti-wrinkle actives, anti-atrophy actives, anti-oxidants, flavanoids, anti-inflammatory agents, anti-cellulite, topical anesthetics, tanning actives, skin soothing actives, skin healing actives, conditioning agents, and mixtures thereof.

23. A skin care composition according to claim 22 wherein the skin care active is selected from the group consisting of niacinamide, panthenol, anti-oxidants, salicylic acid, retinoids, and mixtures thereof.

24. A topical personal care composition comprising:
a) at least one protein;
b) at least one organic powder made from a material selected from the group consisting of boron nitride, cellulose triacetate, ethylene acrylic acid copolymer, mica, sericite, nylon-6, nylon-12, polymethylmethacrylate, aluminum starch octenylsuccinate, polyethylene, polypropylene, silicone resin, silk, talc, and mixtures thereof.
